(19) United States
(12) Patent Application Publication
(10) Pub. No.: US 2017/0368204 A1
(43) Pub. Date: Dec. 28, 2017

(54) POLYMER MICROBUBBLES AS X-RAY DARK FIELD CONTRAST AGENTS

(71) Applicant: UNIVERSITY OF PITTSBURGH - OF HIGHER EDUCATION, Pittsburgh, PA (US)

(72) Inventors: Flordeliza S. Villanueva, Pittsburgh, PA (US); Bin Qin, Pittsburgh, PA (US); Bruce Raymond Whiting, Pittsburgh, PA (US); William R. Wagner, Gibsonia, PA (US); Charles F. McTiernan, Pittsburgh, PA (US)

(21) Appl. No.: 15/543,097

(22) PCT Filed: Jan. 12, 2016
(86) PCT No.: PCT/US2016/013049

§ 371 Date: Jul. 12, 2017

(51) Int. Cl. A61K 49/04; A61B 5/00; A61B 6/00

(52) U.S. Cl. CPC A61K 49/0423; A61B 6/504; A61B 6/484; A61B 5/0097; A61B 6/4035; A61B 6/4291

(57) ABSTRACT

The present invention discloses compositions and methods for clinical imaging. In particular, these compositions and methods provide improvements to cardiovascular imaging. Such improvements are drawn to the creation and use of polymer-based microbubbles comprising metal nanoparticle additives that provide contrast images of highly improve resolution when compared to conventional lipid based microbubbles. For example, the compositions and methods may be used for dark field X-ray scattering contrast images for angiography.

### FIELD OF THE INVENTION

The present invention discloses compositions and methods for clinical imaging. In particular, these compositions and methods provide improvements to cardiovascular imaging, as related to distinguishing between tissue types with less risk of toxicity than conventional contrast agents (e.g., iodine). Such improvements are drawn to the creation and use of polymer-based microbubbles with metal particle additives that provide images of highly improved contrast resolution when compared to conventional lipid based microbubbles. For example, the compositions and methods may be used for dark field X-ray scattering phase contrast or dark field images for angiography.

### BACKGROUND

X-rays are electromagnetic waves of short wavelength that can penetrate and pass through the body due to their short wavelengths, typically between 0.01 and 1 nm. When passing through matter, X-rays are subjected to different types of interactions happening at the atomic level. Only a fraction of the incident X-Rays pass through the body and exit the back surface, the remaining part being absorbed or scattered. Each material is characterized by a macroscopic coefficient describing its ability to stop X-rays that is called “linear attenuation coefficient”, commonly indicated as μ, in [cm⁻¹] units, providing a measure of how many X-rays per unit length are stopped.

Historically, the basic principles of X-ray imaging currently in use in the today's diagnostic practice have remained essentially unchanged since Roentgen's first discovery of X-rays over a hundred years ago. According to this conventional approach, X-rays pass through the body organ or tissue under examination and may exit the back surface or be absorbed by the same. The fraction that emerges is dependent upon the energy of the X-rays, the thickness of the body and the materials present in the body, i.e., tissue, bone, blood and so on.

The basic principles of conventional X-ray imaging and today's medical diagnostic systems rely on X-ray absorption as the sole source of information. Accordingly, differences in absorption produce contrast in the radiographic or tomographic images. In biological tissues calcium absorbs X-rays the most, fat and other soft tissues absorb less, air in lung absorbs the least, and are accordingly recorded in white, grey and black on the X-ray image, respectively. Optimal results are obtained only in distinguishing between hard and soft tissues while the distinction between different kind of soft tissues showing slight differences in density and composition is challenging.

In order to improve X-ray sensitivity and therefore diagnostic accuracy, compositions and methods based upon dark-field X-ray scattering techniques, as opposed to conventional light-field X-ray absorption techniques, are needed in the art.

### SUMMARY OF THE INVENTION

The present invention discloses compositions and methods for clinical imaging. In particular, these compositions and methods provide improvements to cardiovascular imaging. Such improvements are drawn to the creation and use of polymer-based microbubbles with metal particle additives that provide contrast images of highly improved contrast intensity discrimination when compared to conventional lipid based microbubbles. For example, the compositions and methods may be used for dark field X-ray scattering contrast images for angiography.

In one embodiment, the present invention contemplates a method, comprising: a) providing; i) a medical device comprising a microbubble comprising an inner polymer shell and an outer polymer shell wherein said inner polymer shell encapsulates a gas-filled hollow core; ii) at least one metal nanoparticle attached to said outer polymer shell; iii) an X-ray dark field imaging apparatus; and iv) a patient comprising a cardiovascular blood vessel; b) administering said microbubble to said patient with said medical device; c) delivering said microbubble to said cardiovascular blood vessel; and d) imaging said cardiovascular blood vessel with said X-ray dark field imaging apparatus. In one embodiment, the medical device includes, but is not limited to, a catheter, a syringe, an applicator gun, or an endoscope. In one embodiment, the cardiovascular blood vessel is an artery or a vein. In one embodiment, the imaging produces an angiogram. In one embodiment, the imaging produces a venogram. In one embodiment, the cardiovascular blood vessel is a coronary blood vessel. In one embodiment, the cardiovascular blood vessel is a neurovascular blood vessel. In one embodiment, the cardiovascular blood vessel is a peripheral blood vessel. In one embodiment, the blood vessel is a microcirculatory blood vessel. In one embodiment, the at least one metal nanoparticle is selected from the group consisting of at least one gold nanoparticle and at least one iron oxide nanoparticle. In one embodiment, the at least one metal nanoparticle forms a nanoparticle layer. In one embodiment, the at least one metal nanoparticle is attached to said outer polymer shell with a linker. In one embodiment, the at least one metal nanoparticle is covalently attached to said outer polymer shell. In one embodiment, the at least one metal nanoparticle is completely embedded within said outer polymer shell. In one embodiment, the outer polymer shell comprises an amphiphilic biocompatible material. In one embodiment, the amphiphilic biocompatible material is albumin. In one embodiment, the outer polymer shell comprises a first biodegradable polymer. In one embodiment, the inner polymer shell comprises a second biodegradable polymer. In one embodiment, the gas-filled hollow core comprises a gas selected from the group consisting air and nitrogen. In one embodiment, the imaging creates an X-ray dark field image of said cardiovascular blood vessel.

In one embodiment, the present invention contemplates a method, comprising: a) providing; i) a microbubble comprising an inner polymer shell and an outer polymer shell wherein said inner polymer shell encapsulates a gas-filled hollow core; ii) at least one metal nanoparticle attached to said outer polymer shell; iii) an X-ray dark field imaging apparatus; and iv) a target tissue configured for imaging by said X-ray dark field imaging apparatus; b) contacting said microbubble with said target tissue; and c) imaging said target tissue with said X-ray dark field imaging apparatus. In one embodiment, the at least one metal nanoparticle is selected from the group consisting of at least one gold nanoparticle and at least one iron oxide nanoparticle. In one embodiment, the at least one metal nanoparticle forms a nanoparticle layer. In one embodiment, the at least one metal nanoparticle is attached to said outer polymer shell with a linker. In one embodiment, the at least one metal nanoparticle is covalently attached to said outer polymer shell. In one embodiment, the at least one metal nanoparticle is completely embedded within said outer polymer shell. In one embodiment, the outer polymer shell comprises an amphiphilic biocompatible material. In one embodiment, the amphiphilic biocompatible material is albumin. In one embodiment, the outer polymer shell comprises a first biodegradable polymer. In one embodiment, the inner polymer shell comprises a second biodegradable polymer. In one embodiment, the gas-filled hollow core comprises a gas selected from the group consisting air and nitrogen. In one embodiment, the target tissue is within a patient. In one embodiment, the contacting comprises administering said microbubble to said patient. In one embodiment, the imaging creates an X-ray dark field image of said target tissue.

In one embodiment, the present invention contemplates a composition comprising a dual shell polymer microbubble comprising an inner polymer layer and an outer polymer layer, wherein said outer polymer layer comprises at least one metal nanoparticle. In one embodiment, the dual shell polymer further comprises a gas-filled hollow core. In one embodiment, the outer polymer layer further comprises an amphiphilic biocompatible material. In one embodiment, the at least one metal nanoparticle is a metal nanoparticle layer. In one embodiment, the at least one metal nanoparticle is covalently attached to said outer polymer layer. In one embodiment, the at least one metal nanoparticle is completely embedded within said outer polymer layer. In one embodiment, the at least one metal nanoparticle is selected from the group consisting of gold nanoparticle and at least one iron oxide nanoparticle. In one embodiment, the gas-filled hollow core comprises a gas selected from the group consisting of air and nitrogen. In one embodiment, the amphiphilic biocompatible material is a blood compatible material. In one embodiment, the inner polymer layer comprises at least one biodegradable polymer. In one embodiment, the outer polymer layer comprises at least one biodegradable polymer.

### Definitions

The term “polymer contrast microbubble” as used herein, refers to any approximately spherical structure comprising at least one polymer having a diameter of approximately 100 nanometer to 10 microns. Microbubbles may comprise a single layer surface or a bi-layer surface. In conventional usage in clinical ultrasound imaging, the compositions of these microbubbles result in a harmonic oscillation to certain ultrasound frequencies, such that their emissions may be visualized as an image. Alteration of polymer characteristics including, but not limited to, polymer type or concentration, can modify the structural and functional characteristics of a microbubble. Polymers may comprise a linked series of individual components, wherein the components may include, but are not limited to, small organic molecules, amino acids, or nucleic acids. For example, a polymer as contemplated herein includes a peptide or protein and/or an oligonucleotide. A microbubble may contain single or multiple pockets which hold a gas (i.e., for example, an acoustically active gas) or a liquid (i.e., for example, an acoustically active liquid, for example those by Wickline). In general, microbubbles are roughly spherical or irregular shape and have at least one continuous phase comprising a polymer as defined herein. Further, the continuous phase may form one or multiple enclosures capable of entrapping a gas or liquid (i.e., for example, gases or liquids that are acoustically active). Microbubbles may be fabricated by various methods including, but not limited to, double emulsion evaporation or extraction, single emulsion evaporation or extraction, spray drying, freeze spray drying, ultrasound sonication, or microfluidics.

The term “bi-layer microbubble” as used herein, refers to any microbubble comprising a shell having an inner and outer layer. Generally, the outer layer comprises ligands that are amphiphilic and biocompatible (i.e., for example, albumin) and the inner layer comprises biodegradable polymers. The bi-layer is usually between approximately 25-750 nm in width.

The term “controlled fragility microbubble” as used herein, refers to microbubbles within a population of microbubbles having the same wall thickness to diameter ratio.

The term “completely embedded nanoparticle” as used herein refers to any nanoparticle that resides completely within an outer polymer shell of a dual shell microbubble such that the nanoparticle is inaccessible to the surface of the outer polymer shell.

The term “microparticles” as used herein, refer to solid particles ranging from 100 nm to 10 microns in diameter which lack pores such that a gas or liquid is unable to become embedded in the solid continuous phase. Microparticles may be fabricated by various methods including, but not limited to, double emulsion evaporation or extraction, single emulsion evaporation or extraction, spray drying, freeze spray drying, ultrasound sonication, or microfluidics.

The term “target tissue” as used herein, refers to any cardiovascular blood vessel including, but not limited to, an artery or a vein.

The term “contrast agent” as used herein refers to any composition capable of improving the intensity discrimination between at least two different structures having differential densities. Contrast agents contemplated herein include, but are not limited to, polymer microbubbles, stabilized microbubbles, sonicated albumin, gas-filled microspheres, gas-filled liposomes, and gas-forming emulsions.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences. Other concerns are the adverse reaction of the patient to conventional, iodine based contrast agents.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “imaging” as used herein, refers to any technique wherein a visual representation of a cardiovascular blood vessel is created. Such imaging may occur either in vitro, in situ, or in vivo. With specific reference to in vivo imaging, the resolution of the visual representations may be enhanced when performed in combination with a contrast agent.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors

The term “injury” as used herein, denotes a bodily disruption of the normal integrity of tissue structures. In one sense, the term is intended to encompass surgery. In another sense, the term is intended to encompass irritation, inflammation, infection, and the development of fibrosis. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc. Another type of injury is the adverse reaction of a patient to conventional contrast agents containing iodine.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a compound or drug to a patient during a medical procedure or therapy is contemplated herein as a medical device. “Direct medical implants” include, but are not limited to, urinary and intravascular catheters, dialysis shunts, wound drain tubes, skin sutures, vascular grafts and implantable meshes, intraocular devices, implantable drug delivery systems and heart valves, and the like. “Wound care devices” include, but are not limited to, general wound dressings, non-adherent dressings, burn dressings, biological graft materials, tape closures and dressings, and surgical drapes. “Surgical devices” include, but are not limited to, endoscope systems (i.e., catheters, vascular catheters, surgical tools such as scalpels, retractors, and the like) and temporary drug delivery devices such as drug ports, injection needles etc. to administer the medium. A medical device is “coated” when a medium comprising a cytostatic or antiproliferative drug (i.e., for example, sirolimus or an analog of sirolimus) becomes attached to the surface of the medical device. This attachment may be permanent or temporary. When temporary, the attachment may result in a controlled release of a cytostatic or antiproliferative drug.

The term “administered” or “administering” as used herein, refers to any method of providing a composition (i.e., for example, a polymer microbubble comprising a metal nanoparticle) to a patient such that the composition permits visualization of a cardiovascular blood vessel. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “derived from” as used herein, refers to the source of a compound or sequence. In one respect, a compound or sequence may be derived from an organism or particular species. In another respect, a compound or sequence may be derived from a larger complex or sequence.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier” or “pharmaceutically acceptable vehicle”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to “apparent homogeneity” such that there is single protein species (i.e., for example, based upon SDS-PAGE or HPLC analysis). A purified composition is not intended to mean that some trace impurities may remain.

As used herein, the term “substantially purified” refers to molecules that are removed from their natural environment, isolated or separated, and are at least 60% free, preferably 75% free, and more preferably 90% free from other components with which they are naturally associated. The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatibility is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

### BRIEF DESCRIPTION OF THE FIGURES

FIG. 1 presents exemplary data of conventional absorptive X-ray contrast image using polymer microbubbles.

FIG. 2 presents exemplary data showing a relationship of polymer microbubble gas volume fraction (%) and attenuation coefficient.

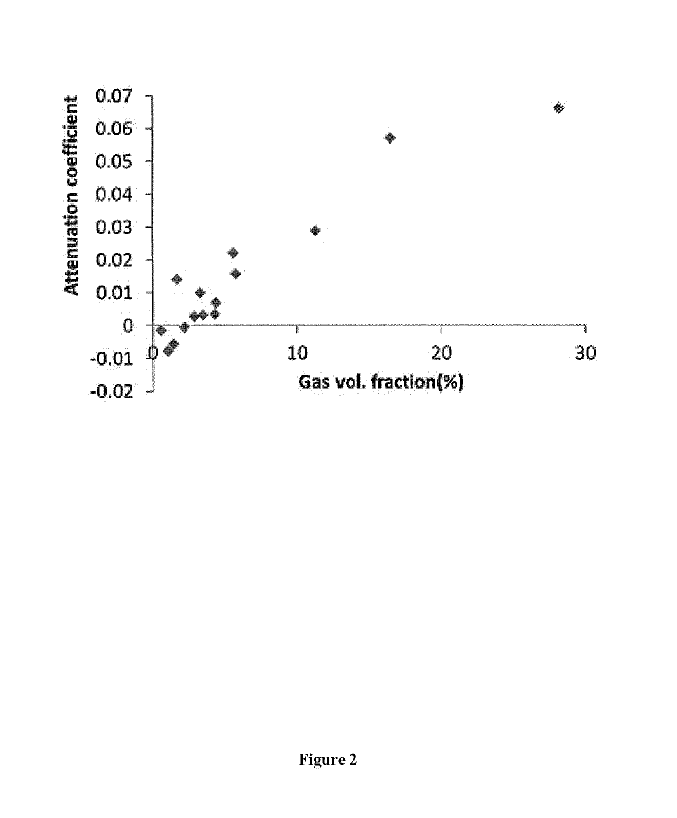

FIG. 3 presents exemplary data of a volumetric size distribution of unfiltered microbubbles, and a size distribution subsequent to suspension filtration.

FIG. 4 shows the resonant frequencies of two microbubble preparations having different wall compositions.

### DETAILED DESCRIPTION OF THE INVENTION

The present invention discloses compositions and methods for clinical imaging. In particular, these compositions and methods provide improvements to cardiovascular imaging. Such improvements are drawn to the creation and use of polymer-based metal nanoparticle microbubbles that provide contrast images of highly improve resolution when compared to conventional lipid based microbubbles. For example, the compositions and methods may be used for dark field X-ray scattering contrast images for angiography.

### II. Conventional X-Ray Imaging

Three types of radiographic images are generally recognized in the art: i) a conventional X-ray image based on X-ray absorption; ii) a differential phase-contrast image based on X-ray refraction; and iii) a dark field image based on X-ray scattering.

A. Conventional X-Ray Imaging

Conventional X-ray diagnostics in clinical use is usually based on X-ray absorption by tissues. Since X-ray absorption depends on tissue density differences, it is difficult to generate optimal contrast for structures with similar densities (for example, a blood vessel and surrounding tissues). Consequently, compounds composed of a high atomic number, such as iodine, are usually used for contrast enhancement in X-ray absorption imaging techniques. Iodine-based X-ray contrast agents have been reported to result in adverse effects such as contrast-induced nephropathy and hypersensitivity reactions. Geenen et al., “Contrast-induced nephropathy: pharmacology, pathophysiology and prevention” Insights Imaging 4(6):811-820; and Nadolski et al., “Contrast alternatives for iodinated contrast allergy and renal dysfunction: options and limitations” J Vasc Surg. 57(2): 593-598. Although carbon dioxide-based contrast agents have been tried as an iodinated contrast agent alternative, these agents also have significant side effects including, but not limited to, air embolisms, and therefore has not been widely clinically accepted. Therefore, there is unmet need for a safer radiographic contrast media and techniques.

B. Conventional Dark Field X-Ray Imaging

An alternative X-ray imaging technique may be dark-field X-ray imaging. This technique measures the radiation scattered by the object instead of recording the attenuation of the X-ray beam (e.g., absorption). Since X-ray scattering is at an atomic/molecular scale of approximately 0.01-0.1 nanometers in size, X-ray dark-field imaging is capable of viewing ultra-fine structure of materials. For example, in vivo multi-contrast X-ray imaging on a small animal has been performed using a compact scanner. X-ray dark-field imaging has been reported to provide a strong contrast signal on the micro-structures of lung (alveoli, ˜10 microns in diameter), which is not possible for an absorption-based X-ray technique. Bech et al., “In-vivo dark-field and phase-contrast x-ray imaging” Sci Rep. 3: 3209.

An x-ray imaging technology has been reported for performing an x-ray dark-field CT imaging comprising an x-ray source, two absorbing gratings G1 and G2, an x-ray detector, a controller and a data processing unit, comprising the steps of: emitting x-rays to the examined object; enabling one of the two absorbing gratings G1 and G2 to perform phase stepping motion within at least one period range thereof; where in each phase stepping step, the detector receives the x-ray and converts it into an electric signal; wherein through the phase stepping of at least one period, the x-ray intensity at each pixel point on the detector is represented as an intensity curve; calculating a second moment of scattering angle distribution for each pixel, based on a contrast of the intensity curve at each pixel point on the detector and an intensity curve without presence of the examined object; taking images of the object at various angles, then obtaining an image with scattering information of the object in accordance with a CT reconstruction algorithm. This method does not contemplate, nor depend upon, any contrast agent to improve the dark-field X-ray image. Huang et al., “X-ray dark-field imaging system and method” European Patent No. 2,453,226.

A method for dark-field imaging has been reported that includes acquiring dark-field image projections of an object with an absorption grating imaging apparatus that includes an x-ray interferometer, applying a pressure wave having a predetermined frequency to the object for each acquired projection, wherein the predetermined frequency is different for each projection, and processing the acquired projections, thereby generating a 3D image of the object. In other words, the method corresponds to an acoustically modulated X-ray dark field tomography technique. This method does not contemplate, nor depend upon, any contrast agent to improve the dark-field X-ray image. Prosaka R., “Dark-Field Imaging” WO 2014/002026.

C. Enhanced Microbubbles

1. Contrast Agents

Conventional contrast agents that have been used in magnetic resonance imaging (MRI), ultrasound (US), conventional X-ray, NMR, Positron emission Tomography (PET), SPECT and Optical Imaging, have been tested for X-ray scattering phenomenon. These contrast agents were evaluated for refractive capabilities using phase-contrast X-ray techniques. To improve the contrast agent refractivity “edge contrast agents” were used that fill an object area with micro/nano scaled edges with contrasting points. This technique generates a so-called “apparent absorption” image that is actually similar to a conventional absorption image but for the presence of the so-called “extinction contrast”. The method simultaneously measures the apparent absorption and the refraction image. This effect increases an image contrast with respect to a pure absorption contrast. Edge contrast agent microbubbles populations consisting of Levovist (galactose/palmitic acid) and Optison were tested for X-ray scattering patterns. Such edge contrast agents were also suggested to include ferromagnetic particles and/or polymer-based microparticles. Nonetheless, a method is not disclosed as useful for collecting images using any dark-field X-ray scattering techniques. Mattiuzzi, M “Contrast enhanced x-ray phase imaging” WO 2004/071535.

Compositions including gadolinium particles encapsulated in albumin microsphere shells have been reported. The composition is suitable for use as a contrast agent with a plurality of imaging modalities, including, for example, ultrasound, magnetic resonance imaging, and computed tomography. The compositions also are useful for therapeutic applications, including neutron capture therapy. McDonald et al., “Contrast agents comprising metal particles encapsulated in microspheres for use in medical diagnostic imaging and therapy” Canadian Patent No. 2,451,852.

2. Nanoparticles

A method of preparing a suspension of microbubbles for use in a carrier liquid, wherein the microbubbles have a gas core and a liquid shell, said liquid shell comprises lipids (e.g., phospholipids, decene/butene copolymers and/or polybutene/polyisobutene copolymers) and/or surfactants in the presence of magnetic nanoparticles has been reported. These lipid-based microbubbles were created to satisfy the following conditions: (i) the force due to buoyancy (FBW) of the microbubble in the carrier liquid is greater than the weight (W) of the microbubble; (ii) the magnetic force (FM) on the microbubble due to a magnetic field applied to the carrier liquid is greater than the combined weight (W) and force due to buoyancy (FBW) of the microbubble; (iii) said magnetic force (FM) on the microbubble is greater than the force due to viscous drag (FD) on the microbubble due to flow of the carrier liquid; and (iv) the scattering cross section (σscat) of the microbubble to ultrasound allows the microbubble to be detectable and rupturable on exposure to ultrasound. Subsequent to administration to a patient the method therefore provides a targeted drug delivery platform upon: i) aggregation of the magnetic nanoparticles at a target site using an externally applied targeted magnetic field; and ii) rupture of the microbubbles, and drug release, using an externally applied targeted ultrasound device. While the method can image the microbubbles themselves, the method does not create dark-field X-ray images. Pankhurst et al., “Magnetic microbubbles, methods of preparing them and their uses” WO 2009/156743.

A targeted microbubble probe for magnetic resonance imaging and blood pressure monitoring has been reported that relates to preparation of a stable coating microbubble material carrying superparamagnetic nanoparticles and a targeted antibody and magnetic resonance monitoring applied to magnetic resonance imaging (MRI) and cardiovascular pressure change. The targeted microbubble probe comprises a gas core, a shell and the superparamagnetic nanoparticles in the shell and targeted molecules on the surface of a membrane shell, wherein the gas comprises paramagnetic gases of nitrogen, argon, oxygen or perfluocarbon; the shell comprises biodegradable poly(N-isopropylamide), polyvinyl alcohol, polylactic acid, chitosan or sodium alga acid; the superparamagnetic nanoparticles comprise ferroferric oxide, gamma-ferric oxide or other ferrite superparamagnetic nanoparticles; and the targeted molecules are RGD and NGR polypeptides of a targeted tumor neovasculature. The method does not utilize the superparamagnetic microbubbles to create dark-field X-ray images. “Targeted micro-bubble probe for magnetic resonance imaging and blood pressure monitoring and preparation method thereof” Chinese Patent Number 101,912,622.

### II. Lipid/Albumin Microbubble X-Ray Dark Field Imaging

There are only two reports using contrast agent microbubbles for X-ray dark-field imaging (infra). In both studies, commercially available lipid-based or albumin-based MBs were used, such as Definity® Microbubbles (Lantheus Medical Imaging Inc.), Optison® Microbubbles (GE Healtcare) and/or SonoVue® Microbubbles (Bracco Altana Pharma), without any modifications. The Definity® microbubble comprises a shell composition of the phospholipids 1,2-dipalmitoyl-sn-glycero-3-phosphate (sodium salt) (DPPA); 1,2-dipalmitoyl-sn-glycero-3-phosphocholine (DPPC), and 1,2-dipalmitoyl-sn-glycero-3-phosphoethanolamine-N-[methoxy(polyethylene glycol)-5000] (sodium salt) (mPEG5000-DPPE) and perflutren as the incorporated gas.

The Definity® vial contains a clear liquid (e.g., water for injection) under a 6.52 mg/mL octafluoropropane headspace that yields perflutren lipid microspheres upon activation with a VialMix® activation. Each mL of the clear liquid contains 0.75 mg lipid blend (consisting of 0.045 mg DPPA, 0.401 mg DPPC, and 0.304 mg mPEG5000-DPPE), 103.5 mg propylene glycol, 126.2 mg glycerin, 2.34 mg sodium phosphate monobasic monohydrate, 2.16 mg sodium phosphate dibasic heptahydrate, and 4.87 mg sodium chloride (pH˜6.2-6.8). After activation, a milky white suspension is formed that contains approximately 1.2×10¹⁰ perflutren lipid microbubbles/mL and about 150 μL/mL octafluoropropane.

The Optison® microbubble shell composition consists of human serum albumin and perflutren as the incorporated gas (Perflutren Protein-Type A Microspheres Injectable Suspension, USP). Optison® is sold as a sterile non-pyrogenic suspension of microbubbles for contrast enhancement during an ultrasound imaging procedure. As sold, each mL of Optison® contains 5.0-8.0×10⁸ protein-type A microspheres, 10 mg Albumin, Human (USP), 0.22±0.11 mg/mL perflutren, 0.2 mg N-acetyltryptophan, and 0.12 mg caprylic acid in 0.9% aqueous sodium chloride (pH: ˜6.4-7.4). The vial headspace is also filled with perflutren gas. The protein in the microsphere shell makes up approximately 5-7% (w/w) of the total protein in the liquid.

The SonoVue® microbubble consists of a shell composition of the phospholipids of distearoylphosphatidylcholine (DSPC), dipalmitoyl phosphatidylglycerol sodium (DPPG) and palmitic acid and sulphur hexafluoride (SF6) as an incorporated gas. SonoVue® is sold as a lyophilized powder where the microbubbles are formed with the addition of a 0.9% NaCl solution. Once formed, SonoVue® microbubbles consist of a sulphur hexafluoride (SF6) core surrounded by a thin and flexible shell of phospholipids. Each vial contains 25 mg sterile pyrogen-free lyophilized white powder comprising Macrogol® 4000 and the head-space of the vial contains the active substance SF6 gas.

It has been reported that lipid and\or albumin-based microbubbles as described above can be used in conjunction with a grating-based X-ray imaging setup to generate contrast-enhanced dark-field X-ray images. For example, contrast agent lipid microbubbles produced dark-field radiography images using a grating-based X-ray dark-field imaging modality where the contrast to noise ratio of these dark-field images were 1.13 to 4.88 times higher than those obtained from absorption-based X-ray. Velroyen et al., “Microbubbles as a scattering contrast agent for grating-based x-ray dark-field imaging” Phys Med Biol. 58(4):N37-N46.

It has also been reported that lipid and/or albumin-based microbubbles can create enhanced contrast image in analyzer-based diffraction enhanced imaging with a synchrotron. This study demonstrated that contrast agent lipid microbubbles can create enhanced contrast in an analyzer-based diffraction enhanced imaging with a synchrotron (using another type of dark-field imaging modality). Arfelli et al., “Microbubbles as x-ray scattering contrast agents using analyzer-based imaging” Phys Med Biol. 55(6):1643-1658.

### III. Polymer-Based Microbubble X-Ray Dark-Field Imaging

There have been no prior reports of polymer-based microbubbles or metal nanoparticle-conjugated microbubbles as dark field scattering radiographic contrast media. Although there are reports of iron oxide nanoparticle-conjugated microbubbles for magnetic resonance-ultrasound dual-modality imaging, the physical principles are completely different from X-ray dark-field imaging.

In one embodiment, the present invention contemplates a composition comprising an X-ray dark field scattering radiographic polymer-based contrast media. This polymer-based contrast media comprises a stabilized gas filled polymer/albumin double shelled microbubble. U.S. Pat. No. 6,193,951 (herein incorporated by reference). In one embodiment, the microbubble further comprises a plurality of metal nanoparticles. In one embodiment, the plurality of metal nanoparticles forms a nanoparticle layer on the microbubble. In one embodiment, the metal nanoparticles include, but are not limited to, gold nanoparticles and/or iron oxide nanoparticles. Although it is not necessary to understand the mechanism of an invention, it is believed that the metal nanoparticles further enhance an X-ray scattering signal.

In one embodiment, the nanoparticles are incorporated within a microbubble polymer shell layer. In one embodiment, the nanoparticles are immobilized on a microbubble polymer shell surface. In one embodiment, the immobilized nanoparticles comprise a covalent linker to the microbubble polymer shell surface. In one embodiment, the immobilized nanoparticles comprise a non-covalent linker to the microbubble polymer shell surface. In one embodiment, the covalent linker and/or non-covalent linker includes, but is not limited to, an ester bond, a disulfide bond, a thioether bond and/or a biotin-streptavidin interaction. Although it is not necessary to understand the mechanism of an invention, it is believed that the covalent linker and/or non-covalent linker can be attached either during microbubble synthesis or once the microbubble has been created.

In one embodiment, the presently claimed microbubbles comprise a tunable polymer contrast media. In one embodiment, the tunable contrast media comprise tunable factors including, but not limited to, microbubble diameter, microbubble lipid shell thickness, metal nanoparticle type, metal nanoparticle size, number of metal nanoparticles incorporated within the microbubble lipid shell, or number of metal nanoparticles immobilized on the microbubble lipid shell surface.

In one embodiment, the present invention contemplates a method comprising: i) administering a microbubble polymer contrast agent comprising a plurality of metal nanoparticles to a patient; and ii) imaging said patient with the contrast agent using dark-field scatter imaging modalities. In one embodiment, the administering is intravascular. In one embodiment, the method further comprises capturing ultra-small angle X-Ray scattering at a gas-to-shell interface. In one embodiment, the method further comprising generating a contrast image based on differences in X-ray scattering capacities.

Polymer-based microbubbles, as disclosed herein, have several unique advantages over microbubbles composed of primarily lipids or primarily albumin:
(1) It is well accepted in the art that polymer microbubbles have improved stability within the cardiovascular system than lipid microbubbles. As the dark field signal strength is associated with microbubble shell-gas interfaces within the X-ray beam path, the microbubble concentration in the blood vessel contributes to the signal strength of dark-field image.
(2) An optimal lipid-based microbubble diameter has been reported for dark-field imaging. Velroyen et al., “Microbubbles as a scattering contrast agent for grating-based x-ray dark-field imaging” Phys Med Biol. 58(4):N37-N46. However, polymer-based microbubbles can be generated under a superior and precise bubble-size control (including diameter and size distribution) than lipid microbubbles and, therefore, comprise a narrower size distribution which achieves an improved dark-field signal.
(3) Metal nanoparticle modifications of polymer-based microbubbles improve X-ray scattering property as compared to a conventional lipid-based microbubble. Consequently, a polymer-based metal nanoparticle microbubble can be used to generate enhanced X-ray dark-field contrast image as compared to a lipid-based metal nanoparticle microbubble.

A. Polymer-Based Bi-Layer Shell Layer Microbubble Compositions

In one embodiment, the microbubbles according to the present invention may have a bi-layered shell. For example, an outer layer of the shell may comprise a biologically compatible material or biomaterial. Although it is not necessary to understand the mechanism of an invention, it is believed that that biological surface material is advantageous to stability as the microbubble surface is exposed to blood and other tissues within the body. The inner layer of a microbubble shell may comprise a biodegradable polymer. For example, the biodegradable polymer comprises a synthetic polymer, which may be tailored to provide the desired mechanical and acoustic properties to the shell or provide drug delivery properties.

In some embodiments, the compositions are polymer-based microbubble ultrasound contrast agents comprising a hollow core of a gas including, but not limited to, air or nitrogen. In some embodiments, compositions are polymer-based rupturable microbubbles (e.g., by a low intensity ultrasound energy). Microbubbles are constructed herein such that the majority of those prepared in a composition will have diameters within the range of about one to ten microns in order to pass through the capillary system of the body.

Since the presently disclosed polymer-based microbubbles have an outer and inner layer, these layers can be tailored to serve different functions. For example, an outer shell which is exposed to the blood and tissues may serve as a biological interface between the microbubbles and the body. Thus, an outer shell comprises a biocompatible material which is typically amphiphilic, that is, has both hydrophobic and hydrophilic characteristics. In other embodiments, blood compatible materials are particularly preferred including, but not limited to, collagen, gelatin or serum albumins or globulins, proteins, glycosoaminoglycans such as hyaluronic acid, heparin and chondroitin sulphate and combinations or derivatives thereof.

In other embodiments, the outer shell layer may comprise a synthetic biodegradable polymer including, but not limited to, polyethylene glycol, polyethylene oxide, polypropylene glycol and combinations or derivatives. As the outer polymer shell layer is typically amphiphilic, as well as having a chemistry which allows charge and chemical modification, this surface versatility allows for modifications including, but not limited to, altering outer shell electrical charge. Electrical charge alterations may be provides by selecting a type A gelatin having an isoelectric point above physiological pH, or by using a type B gelatin having an isoelectric point below physiological pH.

Modifications to polymer-based outer shell layer surfaces may also be chemically modified to enhance biocompatibility, such as by PEGylation, succinylation or amidation, as well as being chemically binding to the surface targeting moiety for binding to selected tissues. The targeting moieties may be antibodies, cell receptors, lectins, selecting, integrins or chemical structures or analogues of the receptor targets of such materials. The mechanical properties of the outer layer may also be modified, such as by cross linking, to make the microbubbles suitable for passage to the left ventricle, to provide a particular resonant frequency for a selected harmonic of the diagnostic imaging system, or to provide stability to a threshold diagnostic imaging level of the ultrasound radiation.

In some embodiments, the polymer-based microbubble comprises an inner shell comprising a biodegradable polymer. In one embodiment, the biodegradable polymer is a synthetic polymer. Although it is not necessary to understand the mechanism of an invention, it is believed that an inner shell provides improved mechanical stabilization properties to the microbubble which are not provided or insufficiently provided by an outer layer alone, or enhances mechanical properties not sufficiently provided by an outer layer alone, without being constrained by surface property requirements. For example, a biocompatible outer layer of a cross-linked proteinaceous hydrogel can be physically supported using a high modulus synthetic polymer as an inner layer. In one embodiment, a high modulus synthetic polymer may be selected for its modulus of elasticity and elongation, which define the desired mechanical properties.

In some embodiments, a biodegradable polymers includes, but is not limited to, polycaprolactone, polylactic acid, polylactic-polyglycolic acid co-polymers, co-polymers of lactides and lactones, such as epsilon-caprolactone, delta-valerolactone, polyalkylcyanoacrylates, polyamides, polyhydroxybutryrates, polydioxanones, poly-beta-aminoketones, polyanhydrides, poly-(ortho)esters, polyamino acids, such as polyglutamic and polyaspartic acids or esters of polyglutamic and polyaspartic acids. Langer, et. al. (1983) Macromol. Chem. Phys. C23, 61-125.

Although it is not necessary to understand the mechanism of an invention, it is believed that a polymer-based microbubble inner layer permits mechanical property modification that cannot be provided by an outer layer alone. In embodiments comprising a polymer-based microbubble ultrasonic contrast agent, an inner layer comprises a thickness which is no larger than is necessary to meet a minimum mechanical constraint. Although it is not necessary to understand the mechanism of an invention it is believed that a combined thickness of the outer and inner layers of the polymer-based microbubble shell depend, in part, on a pre-determined mechanical stability property. Nonetheless, it is believed that a total shell thickness may range between approximately 25 nm to 750 nm.

B. Polymer-Based Bi-Layer Shell Layer Microbubble Synthesis

In some embodiments, polymer-based microbubbles may be prepared by an emulsification process to control the sequential interfacial deposition of shell materials. Due to the amphiphilicity of a material forming an outer layer, stable oil/water emulsions may be prepared having an inner phase to outer phase ratio approaching 3:1, without phase inversion, which can be dispersable in water to form stable organic phase droplets without the need for surfactants, viscosity enhancers or high shear rates.

Two solutions may be prepared: i) an aqueous solution of the outer biomaterial; and ii) a solution comprising an inner layer polymer, an inner layer polymer solvent comprising a relatively volatile water-immiscible liquid, and an inner layer polymer non-solvent comprising a relatively non-volatile water-immiscible liquid. In one embodiment, the inner layer polymer solvent comprises a C5-C7 ester compound (e.g., for example, isopropyl acetate). In one embodiment, the inner layer polymer non-solvent comprises a C6-C20 hydrocarbon (e.g., for example, decane, undecane, cyclohexane and/or cyclooctane).

The two above solutions are combined and agitated so that the inner layer polymer fully dissolves and the two solvents become miscible. The polymer solution (organic phase) is slowly added to the biomaterial solution (aqueous phase) to form a liquid foam. In one embodiment, about three parts of an organic polymer solution having a concentration of about 0.5 to 10 percent of a polymer is added to one part of an aqueous biomaterial solution having a concentration of about 1 to 20 percent of a biomaterial. The relative concentrations of the solutions and the ratio of organic phase to aqueous phase utilized in this step essentially determine the size of the final microbubble and wall thickness. After thorough mixing, the liquid foam is dispersed into water and typically warmed to about 30-35° C. with mild agitation. While not intending to be bound by a particular theory, it is believed that a biomaterial in the foam disperses into the warm water to stabilize an emulsion of the polymer in the organic phase encapsulated within a biomaterial envelope. To render the biomaterial envelope water insoluble, a cross linking agent, such as glutaraldehyde, may be added to the mixture to react with the biomaterial envelope and render it water insoluble, thereby stabilizing the outer shell. Other cross-linking agents may be used including, but not limited to, carbodiimide cross-linkers.

In one embodiment, an inner core is formed comprising a polymer solution, a solvent and a non-solvent. Because the solvent and non-solvent have different volatilities, the more volatile solvent evaporates, or is diluted, and the polymer precipitates in the presence of the less volatile non-solvent. This process forms a film of precipitate at an interface with an inner surface of a biomaterial shell, thus forming an inner shell of a microbubble after the more volatile solvent has been reduced in concentration either by dilution, evaporation or the like. The core of the microbubble then contains predominately an organic non-solvent. The microbubbles may then be isolated by centrifugation, washed, formulated in a buffer system, if desired, and dried. Typically, drying by lyophilization removes not only the non-solvent liquid core but also the residual water to yield gas-filled hollow microbubbles.

In some embodiment, a microbubble surface is modified. In one embodiment, the modification passivates the microbubble surface against macrophages or the reticuloendothelial system (RES) in the liver. This may be accomplished, for example, by chemically modifying the surface of the microbubble to be negatively charged since negatively charged particles appear to better evade recognition by macrophages and the RES than positively charged particles. Also, the hydrophilicity of the surface may be changed by attaching hydrophilic conjugates, such as polyethylene glycol (PEGylation) or succinic acid (succinylation) to the surface, either alone or in conjunction with the charge modification.

A biomaterial microbubble surface may also be modified to provide targeting agents. The surface targeting agents may comprise, for example, antibodies or biological receptors. For example, if a microbubble were modified with targeting agents directed to tumors and were hollow, microbubbles could be used for ultrasound detection to enhance diagnosis of a tumor.

The microbubbles may also be sized or processed after manufacture. This is an advantage over lipid-like microbubbles which may not be subjected to mechanical processing after they are formed due to their fragility. After preparation, but prior to use, the microbubbles may take the form of a lyophilized cake. A later reconstitution of the microbubbles may be facilitated by lyophilization with bulking agents which provide a cake having a high porosity and surface area. The bulking agents may also increase the drying rate during lyophilization by providing channels for the water and solvent vapor to be removed. This also provides a higher surface area which would assist in the later reconstitution. Typical bulking agents are sugars such as dextrose, mannitol, sorbitol and sucrose, and polymers such as PEG's and PVP's.

It is undesirable for microbubbles to aggregate, either during formulation or during later reconstitution of the lyophilized material. Aggregation may be minimized by maintaining a pH of at least one to two pH units above or below the isoelectric point (Pᵢ) of the biomaterial forming the outer surface. A charge on the microbubble surface is determined by the pH of the formulation medium. Thus, for example, if the surface of a biomaterial has a Pᵢ of 7 and the pH of the formulation medium is below 7, the microbubble will possess a net positive surface charge. Alternatively, if the pH of the formulation medium is greater than 7, the microbubble would possess a negative charge. A maximum potential for aggregation exists when a pH of the formulation medium approaches the Pᵢ of a biomaterial used in an outer shell. Therefore, a formulation medium pH at least one to two units above or below the Pᵢ of a microbubble surface minimizes microbubble aggregation. As an alternative, a microbubble may be formulated at or near the Pᵢ with surfactants to stabilize against aggregation.

In one embodiment, an injectable microbubble population formulation comprises a physiologically compatible buffer. Bulking agents may be utilized during microbubble lyophilization to control the osmolality of the final formulation for injection. An osmolality, other than physiological osmolality, may be desirable during lyophilization to minimize aggregation. However, when formulating the microbubbles for use, the volume of liquid used to reconstitute the microbubbles must take this into account.

Other additives may be included in order to prevent aggregation or to facilitate dispersion of the microbubbles upon formulation. Surfactants may be used in the formulation such as poloxomers including, but not limited to, polyethylene, glycol-polypropylene, glycol-polyethylene, and/or glycol block co-polymers. Water soluble polymers also may assist in the dispersion of the microbubbles, such as medium molecular weight polyethylene glycols and low to medium molecular weight polyvinylpyrolidones.

It will be realized that various modifications of the above-described processes may be provided without departing from the spirit and scope of the invention. For example, the wall thickness of both an outer and inner layer may be adjusted by varying concentrations of the components in the microbubble-forming solutions. The mechanical properties of the microbubbles may be controlled, not only by the total wall thickness and thicknesses of the respective layers, but also by selection of materials used in each of the layers by their modulus of elasticity and elongation, and degree of cross-linking of the layers. Upon certain conditions involving rapid deposition of an inner polymer, a very low inner polymer content porosity of the inner polymer shell is observed. The pores range from approximately 0.1 to 2 micron in diameter as observed under electron microscopy.

Mechanical properties of the layers may also be modified with plasticizers or other additives. Adjustment of the strength of the shell may be modified, for example, by the internal pressure within the microbubbles. In particular, by appropriately adjusting the mechanical properties, the microbubbles may be made to remain stable to threshold diagnostic imaging power, while being rupturable by an increase in power and/or by being exposed to its resonant frequency. The resonant frequency can be made to be within the range of transmitted frequencies of diagnostic body imaging systems or can be a harmonic of such frequencies. During the formulation process the microbubbles may be prepared to contain various gases, including blood soluble or blood insoluble gases. It is a feature of the invention that microbubble compositions may be made having a resonant frequency greater or equal to 2 MHz, and typically greater or equal to 5 MHz.

C. Polymer Microbubble-Nanoparticle Complexes

In one embodiment, the present invention contemplates a composition comprising a plurality of polymer microbubble-nanoparticle complexes. See, Table 4.

TABLE 4
Representative polymer microbubble complexes

| | | Concen- | | | |
|---|---|---|---|---|---|
| Sample | Batch number & | tration | Microbubble size (μm) | | |
| # | Composition | (MB/mL) | mean | medium | mode |
| 1 | V010515 - 2 μm | 2.00E9 | 2.661 | 2.539 | 2.577 |
| | N₂/PDLLA/HSA | | | | |
| 2 | Au NP-v010515 -2 μm | 6.40E8 | 2.698 | 2.565 | 2.327 |
| | N₂/PDLLA/HSA/AuNP | | | | |
| 3 | V010515 - 4 μm | 1.80E9 | 3.794 | 3.792 | 4.056 |
| | N₂/PDLLA/HSA | | | | |
| 4 | V091014 | 1.40E9 | 4.372 | 4.314 | 5.088 |
| | N₂/PDLLA/HSA | | | | |
| 5 | V09 | 3.00E9 | 2.690 | 2.483 | 2.577 |
| | N₂/PCL/HSA | | | | |
| 6 | V061215 | 3.95E9 | 3.616 | 3.536 | 4.341 |
| | N₂/PDLLA/HSA | | | | |
| 7 | V062415 | 2.55E9 | 4.138 | 4.065 | 4.700 |
| | N₂/PDLLA/HSA | | | | |
| 8 | V062515 | 2.54E9 | 4.565 | 4.560 | 4.595 |
| | N₂/PDLLA/HSA | | | | |

After preparation, these complexes may be reconstituted, washed to remove the flooded microbubbles and coulter counted prior to use. Although it is not necessary to understand the mechanism of an invention, it is believed that these operations provide a very concentrated microbubble population (See, Samples #1 and #3-#8). In one embodiment, the nanoparticle is a gold nanoparticles (See, Sample #2). In one embodiment, the microbubble is attached to about eight hundred (800) gold nanoparticles. In one embodiment, the polymer microbubbles have dual-layer shell structures. In one embodiment, the dual-layer shell comprises an outer layer comprising glutaraldehyde-fixed albumin. In one embodiment, the dual-layer shell comprises an inner layer comprising a first polymer. In one embodiment, a uniform distribution of a polymer microbubble size was generated using a Shirasu Porous Glass (SPG) model. See, Sample #4 and 6-8. In one embodiment, the dual-layer shell comprises an inner layer comprising a second polymer, wherein said second polymer is different from the first polymer. See, Sample #5. In one embodiment, the dual-layer shell comprises a doubled shell thickness (e.g., two-fold thicker than Samples #1-5). See, Sample #6. In one embodiment, the dual-layer shell comprises a quadruple shell thickness (e.g., four-fold thicker than Samples #1-5). See, Sample #7. In one embodiment, the dual-layer shell comprises an octuple shell thickness (e.g., eight-fold thicker than Samples #1-5). See, Sample #8. Although it is not necessary to understand the mechanism of an invention, it is believed that each of the above polymer microbubble embodiments may have a differential effect on an observed dark field signal generation.

### IV. Angiographic Clinical Applications

In some embodiments the present invention contemplates methods for imaging various diseased and/or injured cardiovascular blood vessels using polymer microbubble-nanoparticle complexes. In one embodiment, the cardiovascular blood vessel is an artery. In one embodiment, the cardiovascular blood vessel is a vein.

Angiography or arteriography is a medical imaging technique used to visualize the inside, or lumen, of blood vessels and organs of the body, with particular interest in the arteries, veins, and the heart chambers. This is traditionally done by injecting a radio-opaque contrast agent into the blood vessel and imaging using X-ray based techniques such as fluoroscopy. The recorded film or image of the blood vessels is called an angiograph, or more commonly, an angiogram. Although, angiography can describe both an arteriogram and a venogram, in its everyday usage, the terms angiogram and arteriogram are often used synonymously, whereas the term venogram is used more precisely.

The term angiography is generally defined as based on projectional radiography; however, the term has been applied to newer vascular imaging techniques such as CT angiography and MR angiography. The term isotope angiography has also been used, although this more correctly is referred to as isotope perfusion scanning.

Depending on the type of angiogram, access to the blood vessels is gained most commonly through the femoral artery, to look at the left side of the heart and at the arterial system; or the jugular or femoral vein, to look at the right side of the heart and at the venous system. Using a system of guide wires and catheters, a type of contrast agent (which shows up by absorbing the x-rays), is added to the blood to make it visible on the x-ray images.

The X-ray images taken may either be still images, displayed on an image intensifier or film, or motion images. For all structures except the heart, the images are usually taken using a technique called digital subtraction angiography or DSA. Images in this case are usually taken at 2-3 frames per second, which allows the interventional radiologist to evaluate the flow of the blood through a vessel or vessels. This technique “subtracts” the bones and other organs so only the vessels filled with contrast agent can be seen. The heart images are taken at 15-30 frames per second, not using a subtraction technique. Because DSA requires the patient to remain motionless, it cannot be used on the heart. Both these techniques enable the interventional radiologist or cardiologist to see stenosis (blockages or narrowings) inside the vessel which may be inhibiting the flow of blood and causing pain.

A. Coronary Angiography

One of the most common angiograms performed is to visualize the blood in the coronary arteries. A long, thin, flexible tube called a catheter is used to administer the X-ray contrast agent at the desired area to be visualized. The catheter is threaded into an artery in the forearm, and the tip is advanced through the arterial system into the major coronary artery. X-ray images of the transient radiocontrast distribution within the blood flowing inside the coronary arteries allows visualization of the size of the artery openings. Presence or absence of atherosclerosis or atheroma within the walls of the arteries cannot be clearly determined.

B. Microangiography

Microangiography is commonly used to visualize tiny blood vessels. Optical coherence tomography (OCT)-based optical microangiography (OMAG) is a high-resolution, noninvasive imaging technique capable of providing three-dimensional in vivo blood flow visualization within microcirculatory tissue beds in the eye. Although the technique has demonstrated early clinical utility by imaging diseased eyes, its limited field of view (FOV) and the sensitivity to eye motion remain the two biggest challenges for the widespread clinical use of the technology. Here, we report the results of retinal OMAG imaging obtained from a Zeiss Cirrus 5000 spectral domain OCT system with motion tracking capability achieved by a line scan ophthalmoscope (LSO). The tracking LSO is able to guide the OCT scanning, which minimizes the effect of eye motion in the final results. LSO tracking can effectively correct the motion artifacts and remove the discontinuities and distortions of vascular appearance due to microsaccade, leading to almost motion-free OMAG angiograms with good repeatability and reliability. Due to the robustness of the tracking LSO, a montage scan protocol can provide unprecedented wide field retinal OMAG angiograms. Zhang et al., “Wide-field imaging of retinal vasculature using optical coherence tomography-based microangiography provided by motion tracking” J Biomed Opt. 20(6):066008 (2015).

C. Neurovascular Angiography

Another increasingly common angiographic procedure is neurovascular digital subtraction angiography in order to visualise the arterial and venous supply to the brain. Intervention work such as coil-embolisation of aneurysms and AVM gluing can also be performed.

Neurovascular angiography can be performed using a device such as an AXERA 2 low-angle vascular access device utilizing a dual arteriotomy mechanism in which the standard access tract is compressed by a vascular sheath inserted over the second, low-angle tract. While this device could be effectively used with 21-gauge micropuncture access, as the micropuncture introducer makes a larger arteriotomy than the 19-gauge needle provided with the AXERA 2 system. A retrospective review was performed on 189 patients who underwent common femoral artery access for diagnostic cerebrovascular angiography using either combined micropuncture and AXERA 2 access or standard access with manual pressure hemostasis. Demographic and procedural data were reviewed along with complications related to vascular access and times to bed elevation, ambulation and discharge. Combined micropuncture and AXERA 2 access was performed on 110 patients and 79 patients had standard access. The AXERA device was successfully used in 91.8% of the cases. Demographic data, anticoagulant use and sheath sizes were similar between both subsets. Use of the AXERA 2 was associated with two bleeding complications (1.8%) compared with 10 (12.7%) with manual pressure hemostasis alone. Institution-specific protocol allowed shorter mean manual compression time, as well as shorter times to ambulation and discharge with the AXERA 2. Use of the AXERA 2 device with micropuncture access did not infer increased bleeding risk than standard arterial access in this patient series. The considerable incidence of device use failures suggests a learning curve associated with its use. Bourgeois et al., “Safety and efficacy of combined micropuncture and shallow angle femoral artery access for neurovascular angiography” J Vasc Access. May 23 (2015).

D. Peripheral Angiography

Angiography is also commonly performed to identify vessel narrowing in patients with leg claudication or cramps, caused by reduced blood flow down the legs and to the feet; in patients with renal stenosis (which commonly causes high blood pressure) and can be used in the head to find and repair stroke. These are all done routinely through the femoral artery, but can also be performed through the brachial or axillary (arm) artery. Any stenoses found may be treated by the use of atherectomy.

Peripheral angiography may be performed using an AVERT™ Contrast Modulation System (AVERT) (Osprey Medical, MN) that is designed to reduce contrast volume administration during angiography. The AVERT provides an adjustable resistance circuit which decreases the pressure head delivering contrast towards the patient. The AVERT has not been previously studied in patients undergoing peripheral digital subtraction angiography (DSA). Patients undergoing lower extremity DSA (diagnostic or intervention, sheath or catheter) were studied using the AVERT system. The following variables were recorded for each injection: starting control syringe contrast volume, contrast volume injected towards patient, contrast volume returned to AVERT reservoir, net contrast administered to the patient and % savings. The AVERT resistance was adjusted manually based on operator's discretion-balancing image quality and contrast savings. It was found that about 408 DSA angiographic sequences were obtained in 22 patients undergoing 29 procedures. Almost 68% of the patients had chronic kidney disease. An 82% presented with critical limb ischemia, 18% had claudication. There was an overall 37%±14% savings of contrast (31% for diagnostic DSA, 40% for interventional procedures). Overall 91% of all images were acceptable for clinical decision making. Specifically, 94% of diagnostic and 87% of interventional images were acceptable. Injection through a 4 Fr catheter (77% acceptable) resulted in poorer image quality as compared to a 5 Fr catheter (96% acceptable). Image quality for 5, 6, and 7 Fr sheath injections was 86%, 91%, 98%, respectively. The bench model of peripheral angiography demonstrated a significant reduction in reflux of contrast proximal to the end of the catheter without loss of antegrade image quality—confirming the in vivo findings. This report demonstrates that the use of the AVERT device during peripheral angiography results in significant contrast savings without compromising image quality. Prasad et al., “The use of the AVERT system to limit contrast volume administration during peripheral angiography and intervention” Catheter Cardiovasc Interv. 86(7):1228-1233. (2015).

### VII. Pharmaceutical Compositions And Formulations

The present invention further provides pharmaceutical compositions comprising a polymer microbubble-nanoparticle complex. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### VIII. Kits

In another embodiment, the present invention contemplates kits for the practice of the methods of this invention. The kits preferably include one or more containers containing a polymer microbubble-nanoparticle complex comprising an acoustically active gas. The kit can optionally include a pharmaceutically acceptable excipient and/or a delivery vehicle. The reagents may be provided suspended in the excipient and/or delivery vehicle or may be provided as a separate component which can be later combined with the excipient and/or delivery vehicle.

The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in: i) delivering a polymer microbubble-nanoparticle complex to a biological tissue; and iii) imaging the biological tissue with the delivered polymer microbubble-nanoparticle complex. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXPERIMENTAL

The following examples are provided by way of illustration but are not intended to limit the invention in any way.

### Example 1

### Preparation of Gelatin Polycaprolactone Microbubbles

A solution of 1.0 gms gelatin (275 bl, isoelectric point of 4.89) dissolved in 20 ml deionized water was prepared at approximately 60 C. Native pH of the solution was 5.07. Separately, 1.0 gms polycaprolactone (M.W. 50,000) and 6.75 ml cyclooctane was dissolved in 42 ml isopropyl acetate with stirring at approximately 70 C. After cooling to 37 C, the organic mixture was then slowly incorporated into the gelatin solution maintained at 30 C and under moderate shear mixing using a rotary mixer. Once the organic phase was fully incorporated, the mixing rate was increased to 2,500 rpm for 5 minutes and then stirred at low shear for an additional 5 minutes. The resulting o-w emulsion was then added with stirring to 350 ml deionized water maintained at 30 C and containing 1.2 ml 25% glutaraldehyde Immediately after the addition of the emulsion, the bath pH was adjusted to 4.7. After 30 minutes, the pH was adjusted to 8.3. Low shear mixing was continued for approximately 2½ hours until the isopropyl acetate had completely volatilized. Polyoxamer 188 in the amount of 0.75 gm was then dissolved into the bath. The resulting microbubbles were retrieved by centrifugation and washed 2 times in an aqueous solution of 0.25% polyoxamer 188.

Microscopic inspection of the microbubbles revealed spherical capsules having a thin-walled polymer shell encapsulating a liquid organic core. Staining the slide preparation with Coomassie blue G indicated the presence of an outer protein layer uniformly surrounding the polymer shell.

The particle size spectrum was determined using a Malvern Micro. Median diameter was 4.78 microns with a spectrum span of 0.94.

### Example 2

### Preparation of a Contrast Agent Formulation

A quantity of microbubbles prepared in accordance with Example 1 were suspended into an aqueous solution of 25 mM glycine, 0.5% pluronic f-127, 1.0% sucrose, 3.0% mannitol, and 5.0% PEG-3400. The suspension was then lyophilized. The resulting dry powder was reconstituted in deionized water and examined under the microscope to reveal that the microbubbles now contained a gaseous core. Staining the preparation with Coomassie blue G confirmed that the outer protein layer surrounding the capsules was intact and had survived the lyophilization process.

Echogenicity was confirmed by insonating at both 2½ and 5 MHz a quantity of lyophilized microbubbles dispersed in 120 ml deionized water. Measurement was taken at least 15 minutes after dispersion of the microcapsules to insure that the back scattered signal was due solely from the gas contained within the microbubbles. The B mode display showed a high contrast indicating that the microbubbles were gas filled.

### Example 3

### Preparation of Gelatin Polylactide Microbubbles

A solution of 1.2 gm gelatin (225 bloom, isoelectric point of 5.1) dissolved in 20 ml deionized water was prepared at approximately 50 C. Solution pH was adjusted to 6.1 using 1 M NaOH. Separately, 0.07 gms paraffin, 4.5 ml decane, and 0.69 gms poly DL-lactide (inherent viscosity of 0.69 dL/gm in CHCl₃ @ 30° C.) was dissolved into 37 ml isopropyl acetate. The organic mixture was then slowly incorporated into the gelatin solution which was being maintained at 30 C under moderate shear mixing using a rotary mixer. Once the organic phase was fully incorporated, the mixing rate was increased to 2,000 rpm for 2 minutes and then reduced to approximately 1,000 rpm for 4 minutes. The resulting liquid foam was mixed into 350 ml deionized water maintained at 30° C. and 1 ml 25% glutaraldehyde was then added dropwise. Rotary mixing was continued for approximately 3 hours until the isopropyl acetate had volatilized. The resulting microbubbles were retrieved by centrifugation and washed 2 times in an aqueous solution of 0.25% pluronic f-127.

Microscopic inspection revealed hollow spherical microbubbles having an outer protein layer and an inner organic liquid core.

The microbubbles were lyophilized and tested in a manner similar to Example 2. The results confirmed that the microbubbles contained a gaseous core and were strongly echogenic.

### Example 4

### Preparation of Gelatin Polycaprolactone Microbubbles

A solution of 1.0 gm gelatin (225 bloom, isoelectric point of 5.1) dissolved in 20 ml deionized water was prepared at approximately 60 C. Solution pH was 4.8. Separately, 0.57 gms polycaprolactone (M.W. 50,000) was dissolved into 1.72 ml tetrahydrofuran. To this was added with stirring a mixture of 0.07 gms paraffin, 0.475 gm triethyl citrate, 4.5 ml cyclooctane, and 42 ml isopropyl acetate. The organic mixture was then slowly incorporated into the gelatin solution which was maintained at 30 C and under moderate shear mixing using a rotary mixer. Once the organic phase was fully incorporated, the mixing rate was increased to 4,700 rpm for 2 minutes and then reduced to 2,000 rpm for 4 minutes. The resulting liquid foam was then added with stirring to 350 ml of 30 C deionized water. To crosslink the gelatin, 1 ml of 25% glutaraldehyde was added dropwise. Mixing was continued for approximately 3 hours until the isopropyl acetate had volatilized. The resulting microbubbles were retrieved by centrifugation and washed 2 times in a 0.25% pluronic f-127 solution.

Microscopic inspection revealed discrete hollow spherical polymer microbubbles having an outer protein layer and an inner organic liquid core.

The microbubbles were lyophilized and tested in a manner similar to Example 2. The results confirmed that the microbubbles contained a gaseous core and were strongly echogenic.

### Example 5

### Preparation of Gelatin Polycaprolactone Microbubbles with Carbodiimide Cross-Linking

A solution of 1.0 grams gelatin (225 bloom, isoelectric point of 5.1) dissolved into 20 ml deionized water was prepared at approximately 60 C. Solution pH was adjusted to 5.5 with 1 M NaOH. Separately, 0.85 gms polycaprolactone (M.W. 80,000) was dissolved in 2.5 ml tetrahydrofuran. To this was added with stirring a mixture of 0.07 gms paraffin, 4.5 ml cyclooctane and 42 ml isopropyl acetate. The organic mixture was then slowly incorporated into the gelatin solution which was maintained at 30° C. and under moderate shear mixing using a rotary mixer. Once the organic phase was fully incorporated, the mixing rate was increased to 3,500 rpm for 6 minutes and then reduced to 3,000 rpm for 4 minutes. The resulting liquid foam was then dispersed with low shear mixing into 350 ml of a 0.5 M NaCl solution maintained at 30° C. Gelatin crosslinking was accomplished by the slow addition of 200 mg of 1-ethyl-3-(3-dimethylamino-propyl)carbodiimide dissolved in 3.0 ml deionized water. Mixing was continued for approximately 3 hours until the isopropyl acetate had volatilized. The resulting microbubbles were retrieved by centrifugation and washed 2 times in an aqueous solution of 0.25% Pluronic f-127.

Microscopic inspection revealed discrete hollow spherical polymer microbubbles having an outer protein layer and an inner organic liquid core.

### Example 6

### Preparation of Surface PEGylated Microbubbles

Microbubbles were prepared in accordance with Example 1. After centrifugation the cream (approximately 15 ml) was retrieved and dispersed into a solution of 65 ml deionized water, 0.50 gms methoxy-PEG-NCO (M.W. 5000), and 0.50 ml triethylamine. After allowing the mixture to react overnight at room temperature and with mild agitation, the capsules were retrieved by centrifugation and washed 3 times in a neutrally buffered solution of 0.25% Pluronic F-127.

### Example 7

### Biotin Modification of a Microbubble Surface

Microbubbles were prepared in accordance with Example 11. 50 mL (approximately 1.5×109 microsphere per ml) microbubble containing cream was centrifuged and resuspended in 48 mL PBS (pH=7.4) containing 2 mM EDTA, To this was added a 2 ml solution containing 100 gm maleimide-PEG2-biotin. The mixture was allowed to react at room temperature with mild agitation for 2.5 hours. At the end of this period, the microbubbles were retrieved by centrifugation and washed 2 times in a 0.25% solution of poloxamer 188. The suspension was formulated with a glycine/PEG 3350 excipient solution, then lyophilized. The resulting dry cake was reconstituted with deionized water and examined under the microscope to reveal that the microbubbles were spherical, discrete, and contained a gaseous core.

### Example 8

### Conjugation Iron Oxide Nanoparticle to a Microbubble Surface

The water-reconstituted biotinylated microbubbles were prepared in accordance with Example 7. 200 μl microbubble was centrifuged and resuspended in 200 μl PBS (pH=7.4). To this was added a 40 μl solution containing 0.2 mg streptavidin. The mixture was incubated at room temperature with mild agitation for 1 hour. At the end of this period, the microbubble was washed 3 times in PBS (pH=7.4). 200 μl streptavidinylated microbubble (approximately 4.5×108 microsphere per ml) was added into 200 μl biotinylated iron oxide nanoparticle (diameter: 30 nm, 0.3 mg Fe/mL). The mixture was incubated at room temperature with mild agitation for 1 hour. The iron oxide nanoparticle conjugated microbubbles were retrieved by centrifugation and washed 4 times with PBS (pH=7.4).

### Example 9

### Preparation of Albumin Polycaprolactone Microbubbles

A 6% aqueous solution was prepared from a 25% solution of USP grade human serum albumin (Alpha Therapeutic Corp) by dilution with deionized water. The solution was adjusted to a pH of 3.49 using 1 N HCl. Separately, 8 parts by weight polycaprolactone (M.W. 50,000) and 45 parts cyclooctane were dissolved in 300 parts isopropyl acetate at approximately 70° C. Once dissolution was complete, the organic solution was allowed to cool to 37° C. With mild stirring, 42.5 gm of the prepared organic solution was slowly incorporated into 25.0 gm of the albumin solution while the mixture was maintained at 30° C. The resulting coarse o-w emulsion was then circulated through a stainless steel sintered metal filter element having a nominal pore size of 7 microns. Recirculation of the emulsion was continued for 8 minutes. The emulsion was then added with stirring to 350 ml deionized water maintained at 30° C. and containing 1.0 ml of 25% glutaraldehyde. During the addition, the pH of the bath was monitored to insure that it remained between 7 and 8. Final pH was 7.1. Low shear mixing was continued for approximately 2½ hours until the isopropyl acetate had completely volatilized. Poloxamer 188 in the amount of 0.75 gm was then dissolved into the bath. The resulting microbubbles were retrieved by centrifugation and washed 2 times in an aqueous solution of 0.25% poloxamer.

Microscopic inspection of the suspension revealed spherical particles having a thin-walled polymer shell with an outer protein layer and an organic liquid core. The peak diameter as, determined by the Malvern Micro particle size analyzer, was 4.12 microns.

The suspension was then lyophilized in a manner similar to that described in Example 2. The resulting dry cake was reconstituted with deionized water and examined under the microscope to reveal that the microbubbles were spherical, discrete, and contained a gaseous core.

### Example 10

### Protein Content of Microbubbles

Microbubbles were prepared in accordance with Example 9. After centrifugation approximately 1 ml of the microbubble containing cream was retrieved and diluted 10 to 1 using deionized water. From the diluted cream, 20 microliter samples were then prepared in triplicate at 1×, 2×, and 4× dilutions with deionized water. Protein content of the samples were determined using a Pierce calorimetric BCA assay and a bovine serum albumin standard. Average total protein of the diluted cream was determined to be 0.441 mg/ml. To determine the total dry weight of the diluted cream, 2 ml were dried in a 40° C. oven until no further weight change was observed (approximately 16 hours). The average weight of 4 replicates was 6.45 mg/ml. The percent dry weight of protein which can be used as a measure of the ratio of the protein outer layer to the polymer inner layer of the microcapsule wall can be determined with the following formula.

The percent dry weight of protein was calculated to be 6.8%.

### Example 11

### Preparation of Albumin Polylactide Microbubbles

A 6% aqueous solution was prepared from a 25% solution of USP grade human albumin by dilution with deionized water. Ion exchange resin (AG 501-X8, BioRad Laboratories) was then added to the solution at a ratio of 1.5 gm resin to 1.0 gm dry weight of albumin. After 3 hours the resin was removed by filtration and the pH of the solution was adjusted from 4.65 to 5.5 Separately, 0.41 gm d-1 lactide (0.69 dL/gm in CHCl₃: at 30° C.) and 5.63 gm cyclooctane were dissolved in 37.5 gm isopropyl acetate. The organic solution was then slowly incorporated into 25.0 gm of the prepared albumin solution with mild stirring while the mixture was maintained at 30° C. The resulting coarse o-w emulsion was then circulated through a stainless steel sintered metal filter element having a nominal pore size of 7 microns. Recirculation of the emulsion was continued for 8 minutes. The emulsion was then added with stirring to 350 ml deionized water maintained at 30 C and containing 1.0 ml of 25% glutaraldehyde. During the addition, the pH of the bath was monitored to insure that it remained between 7 and 8. Final pH was 7.0. Low shear mixing was continued for approximately 2½ hours until the isopropyl acetate had completely volatilized. Polyoxamer 188 in the amount of 0.75 gm was then dissolved into the bath. The resulting microspheres were retrieved by centrifugation and washed 2 times in an aqueous solution of 0.25% polyoxamer.

Microscopic inspection revealed hollow spherical polymer microbubbles having an outer protein layer and an inner organic liquid core. The suspension was formulated with a glycine/PEG 3350 excipient solution, then lyophilized. The resulting dry cake was reconstituted with deionized water and examined under the microscope to reveal that the microbubbles were spherical, discrete, and contained a gaseous core.

### Example 12

### PEG Modification of a Microbubble Surface

Microbubbles were prepared in accordance with Example 9. After centrifugation, 4 ml of the microbubbles containing cream (approximately 11 ml total yield) was resuspended in 31 ml deionized water. To this was added a 10 ml solution containing 0.3 gm methoxy-peg-NCO 5000 and the pH was adjusted to 8.7. The mixture was allowed to react at room temperature with mild agitation for 4½ hours. At the end of this period the pH was measured to be 7.9. The microbubbles were retrieved by centrifugation and washed 2 times in a 0.25% solution of polyoxamer 188. The suspension was formulated with a glycine/PEG 3350 excipient solution, then lyophilized. The resulting dry cake was reconstituted with deionized water and examined under the microscope to reveal that the microbubbles were spherical, discrete, and contained a gaseous core.

### Example 13

### Post-Fabrication, Modification of Size Distribution

A quantity of microbubbles were prepared in accordance with Example 1 with procedures modified to provide a broadened size spectrum. After washing and retrieval by centrifugation roughly half the microbubble containing cream was diluted to 125 ml with a 0.25% solution of polyoxamer 188. The suspension was then filtered using a 5 micron sieve type pc membrane filter (Nucleopore) housed in a stirred cell (Amicon). The retentate was discarded while the permeate was again filtered using a 3 micron sieve type filter in the stirred cell system until the retentate volume reached approximately 20 ml. The retentate was diluted to a volume of 220 ml using 0.25% polyoxamer 188 solution. The 3 micron filtration process was repeated until the retentate volume was again approximately 20 ml.

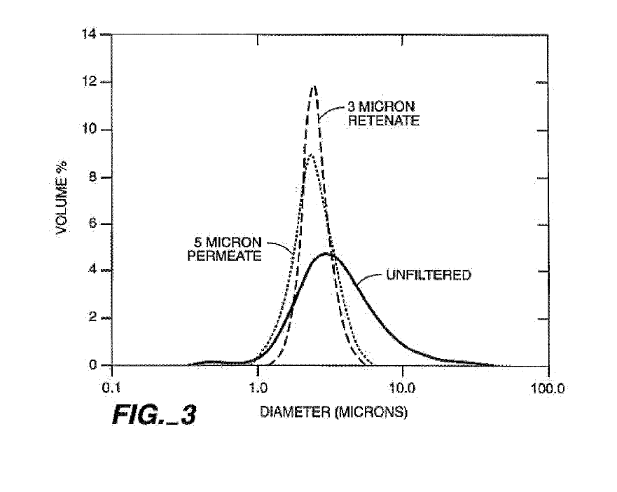

FIG. 3 provides a comparison of the volumetric size distribution of the unfiltered microbubble suspension with the 5 micron permeate and the 3 micron retentate. The results, derived from a Malvern Micro particle size analyzer show a stepwise narrowing of the size spectrum toward a specific size range defined by the pore size of the filters used.

### Example 14

### Absorption Based X-Ray Image of Polymer Microbubble (MB)

1 cc syringes (OD=4.9 mm) filled with various samples (MB, saline or air) were fixed on the bottom of a water bath (water depth: 3-4 cm). The absorption based X-ray image was obtained. The attenuation coefficient of samples was calculated using following formula where in:

$$= ( log \frac{unattenuated ( sample )}{attuenated ( sample )} - log \frac{unattenuated ( s aline )}{attenuated ( saline )} ) / T$$

m: attenuation coefficient

### T: Syringe I.D.

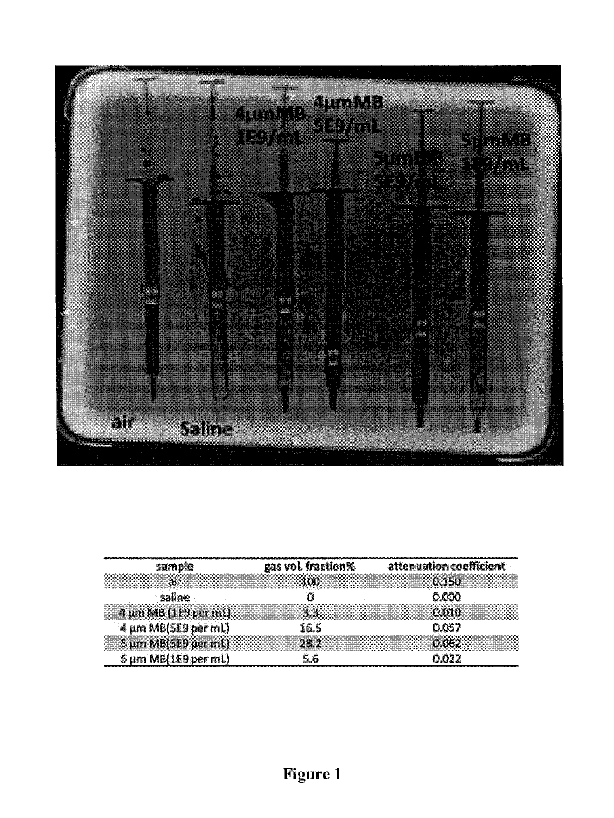

These data (See, FIG. 1) will show less contrast discrimination as compared a dark field (refraction-based) X-Ray Image of polymer microbubbles.

### Example 15

### Dye Loading of Albumin Polylactide Microbubbles

A lyophilized cake in a 10 ml serum vial, composed of excipient and lactide-containing microbubbles prepared in accordance with Example 11 was placed into a 50 ml centrifuge tube. Enough isopropyl alcohol was added to cover the cake and it was allowed to soak for 30 seconds. Aqueous Pluronic F68 solution (0.25% w/w) was added to fill the tube. After centrifuging, the supernatant was removed and another rinse performed. A saturated, filtered solution of rhodamine B was added to the microbubbles and allowed to soak overnight. Under the microscope, the microbubbles appeared filled with dye solution. A dye saturated F68 solution was made to use as a lyophilization excipient. Four ml of the excipient was combined with the approximately 2 ml of microcapsule containing solution and the resulting mixture was split between two 10 ml serum vials. The vials were frozen at −80° C. and lyophilized in a FTS tray dryer. Both vials were purged with perfluorobutane gas by five pump-down purge cycles with a vacuum pump. Observation showed some microbubbles that were half full of red solution and half full of gas. There was no obvious leakage of the dye from these microbubbles during observation. The microbubbles were rinsed with four, 20 ml portions of F68 solution on a vacuum filter. The microbubbles were placed in a cuvette, centrifuged, and an initial spectra was taken. The cuvette was sonicated in an ultrasonic bath, centrifuged, and another spectra taken:

| Abs. Initial (553-800) | Abs. Sonicated (553-800) |
|---|---|
| 1.164 | 1.86 |

The higher absorption after sonication indicates that encapsulated dye was released upon insonation of the microbubbles.

### Example 16

### Preparation of Wall Modified Albumin Polycaprolactone Microbubbles

Albumin coated microcapsules were prepared in accordance with Example 9 with the exception that 0.20 gm paraffin was also dissolved into the organic solution along with the polycaprolactone and the cyclooctane.

Microscopic inspection of the finished microbubble suspension revealed spherical particles having a morphology and appearance virtually identical to those prepared without the addition of paraffin.

### Example 17

### Dye Loading of Human Serum Albumin Polycaprolactone Microbubbles

A lyophilized cake in, a 10 ml serum vial, composed of excipient and paraffin-containing microbubbles prepared in accordance with example 16 was placed into a 50 ml centrifuge tube. The cake was covered with methanol and allowed to soak for 30 seconds. The tube was then filled with an aqueous solution of 0.25% (w/w) Pluronic F68, gently mixed, and centrifuged in order to precipitate the now fluid-filled microcapsules. The supernatant was removed and the tubes were again filled with pluronic solution. The microbubbles were resuspended by vortexing and again centrifuged. After removing the supernatant solution, two ml of a saturated, filtered solution of brilliant blue G dye in 0.25% (w/w) aqueous F68 was added. The microbubbles were allowed to soak for approximately 72 hours. Microscopic examination revealed 90-95% of the microbubbles to be filled with dye solution. A lyophilization excipient was prepared. Four ml of the excipient was added to the microbubble solution and mixed by vortexing. Two 10 ml serum vials were filled with 3 ml each of solution and frozen at −80° C. The vials were lyophilized on a FTS flask lyophilizer. Both vials and a portion of deionized water were purged with perfluorobutane for 10 minutes. Both vials were reconstituted with deionized water and rinsed with two 40 ml portions of 0.25% (w/w) F68 solution on a vacuum filter. The resulting microbubble solution was split into two 3 ml portions. One portion was sonicated in an ultrasonic bath to rupture the bubbles. Both portions were diluted 1/10 with F63 solution and placed into UV-visible cuvettes. The cuvettes were centrifuged and a visible spectra was taken: i) Sonicated=0.193; ii) Non-sonicated=0.136. The higher absorption after sonication indicates that encapsulated dye was released upon insonation of the microcapsules.

### Example 18

### Acoustic Resonance of Microbubbles

To demonstrate a method of acoustically tuning the microbubble construct, microbubbles were prepared in accordance with Examples 9 and 16 and reconstituted with deionized water and compared for their acoustic properties using procedures described as follows:

Two matched 5 MHz transducers were placed in a tank filled with degassed water facing one another. Water depth was approximately 3 inches. The transducers, one an emitter and the other a receiver, were positioned 6 inches apart to maximize the received signals. A 2 inch diameter, 2 cm wide circular chamber was placed between the two transducers with the mid chamber position at 3 inches from the emitter. The two circular faces of the chamber were covered with 3 mil polyethylene film and the chamber was then filled with degassed water. Sound waves readily propagated from the emitter through the chamber to the receiver. The sound source was set to Gaussian Noise with 10 Volt peak to peak amplitude output from the ultrasound generator. The receiver signal is amplified with a 17 dB preamp and an oscilloscope. The oscilloscope digital electronics can perform Fast Fourier Transforms (FFT) of the received wave forms and display these distributions. After baseline readings were made, test microbubble contrast materials were delivered within the chamber via hypodermic syringe and thoroughly mixed therein by pumping the syringe. During post-test evaluation, the FFT data was converted into the Transfer Function of the test agent.

The Transfer Function (TF) is determined by dividing the bubble transmission spectral data by the spectral data without bubbles, i.e.:

TF=T(f)_{with bubbles}/T(f)_{no bubbles}

where T(f) is requested by the FFT. The contrast agent selectively attenuates sound waves depending upon its spectral distribution, i.e. more sound energy is absorbed at or near bubble resonance than off-resonance. Thus the procedure can be used to assess the resonant spectral distribution of the agent.

Data derived from the two agents with nearly identical size distribution but different inner shell thickness were collected on the same day with the same equipment set at the same settings. Everything else was held constant for a variety of agent dosages.

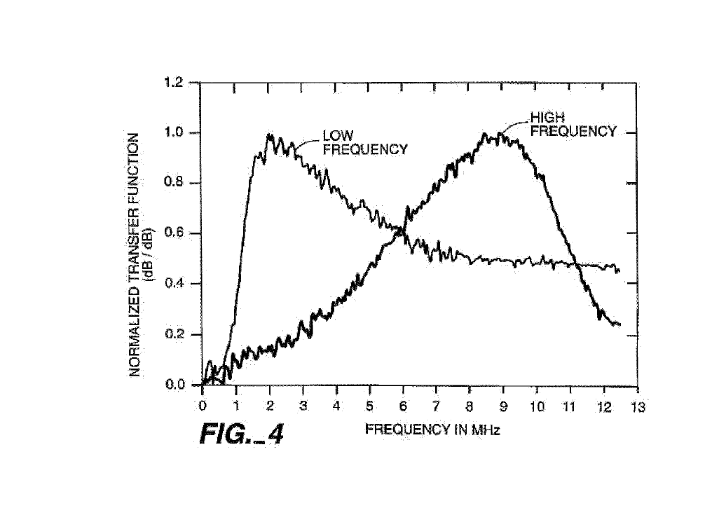

Normalization of the spectra was performed by dividing the spectral array by the minimal value. Thus the peak value becomes unity and when plotted on the same graph it becomes quite easy to differentiate the two graphs. These normalized data are presented in FIG. 4.

Inspection of the results shown in FIG. 4 clearly show that when shell wall compliance is increased, the resonant frequency can be made to shift from 2.3 MHz to 8.9 MHz. Thus, the resonant frequency of an agent can be controlled by controlling the wall composition and thickness.

### Example 18

### Imaging of Coronary Artery Angiography

A polymer microbubble-nanoparticle complex population was prepared as described herein as exemplified in Table 4. Control lipid microbubble-nanoparticle complex populations and lipid microbubble populations were prepared using standard techniques.

To perform coronary artery angiography, 6 rats may be subjected to a 15 min coronary artery occlusion. After 4 hrs reflow, each rat receives separate i.v. boluses of a polymer microbubble-nanoparticle complex population, a lipid microbubble-nanoparticle complex population or a lipid microbubble population. Dark field X-ray imaging is performed 3 and 3.5 min later.

Polymer microbubble-nanoparticle complex populations provide a statistically significant higher resolution of coronary artery angiographic images demonstrating tissue injury (e.g., for example, stenosis) than either the lipid microbubble-nanoparticle population or the lipid microbubble population.

We claim:

1. A method, comprising:
a) providing;
i) a medical device comprising a microbubble comprising an inner polymer shell and an outer polymer shell wherein said inner polymer shell encapsulates a gas-filled hollow core;
ii) at least one metal nanoparticle attached to said outer polymer shell;
iii) an X-ray dark field imaging apparatus; and
iv) a patient comprising a cardiovascular blood vessel;
b) administering said microbubble to said patient with said medical device;
c) delivering said microbubble to said cardiovascular blood vessel; and
d) imaging said cardiovascular blood vessel with said X-ray dark field imaging apparatus.

2. The method of claim 1, wherein said medical device is selected from the group consisting of a catheter, a syringe, an applicator gun, or an endoscope.

3. The method of claim 1, wherein said cardiovascular blood vessel is an artery or a vein.

4. The method of claim 1, wherein said imaging produces an angiogram.

5. The method of claim 1, wherein said imaging produces a venogram.

6. The method of claim 1, wherein said cardiovascular blood vessel is selected from the group consisting of a coronary blood vessel, a neurovascular blood vessel, a peripheral blood vessel, and a microcirculatory blood vessel.

7. The method of claim 1, wherein said at least one metal nanoparticle is selected from the group consisting of at least one gold nanoparticle and at least one iron oxide nanoparticle.

8. The method of claim 1, wherein said at least one metal nanoparticle fat is a nanoparticle layer.

9. The method of claim 1, wherein said at least one metal nanoparticle is attached to said outer polymer shell with a linker.

10. The method of claim 1, wherein said at least one metal nanoparticle is covalently attached to said outer polymer shell.

11. The method of claim 1, wherein said at least one metal nanoparticle is completely embedded within said outer polymer shell.

12. The method of claim 1, wherein said outer polymer shell comprises an amphiphilic biocompatible material.

13. The method of claim 12, wherein said amphiphilic biocompatible material is albumin

14. The method of claim 1, wherein said outer polymer shell comprises a first biodegradable polymer.

15. The method of claim 1, wherein said inner polymer shell comprises a second biodegradable polymer.

16. The method of claim 1, wherein said gas-filled hollow core comprises a gas selected from the group consisting air and nitrogen.

17. The method of claim 1, wherein said imaging creates an X-ray dark field image of said cardiovascular blood vessel.

18. A method, comprising:
a) providing;
i) a microbubble comprising an inner polymer shell and an outer polymer shell wherein said inner polymer shell encapsulates a gas-filled hollow core;
ii) at least one metal nanoparticle attached to said outer polymer shell;
iii) an X-ray dark field imaging apparatus; and
iv) a target tissue configured for imaging by said X-ray dark field imaging apparatus;
b) contacting said microbubble with said target tissue; and
c) imaging said target tissue with said X-ray dark field imaging apparatus.

19. The method of claim 18, wherein said at least one metal nanoparticle is selected from the group consisting of at least one gold nanoparticle and at least one iron oxide nanoparticle.

20. The method of claim 18, wherein said at least one metal nanoparticle is attached to said outer polymer shell with a linker.

21. The method of claim 18, wherein said at least one metal nanoparticle is covalently attached to said outer polymer shell.

22. The method of claim 18, wherein said at least one metal nanoparticle is completely embedded within said outer polymer shell.

23. The method of claim 18, wherein said outer polymer shell comprises an amphiphilic biocompatible material.

24. The method of claim 23, wherein said amphiphilic biocompatible material is albumin.

25. The method of claim 18, wherein said outer polymer shell comprises a first biodegradable polymer.

26. The method of claim 18, wherein said inner polymer shell comprises a second biodegradable polymer.

27. The method of claim 18, wherein said gas-filled hollow core comprises a gas selected from the group consisting air and nitrogen.

28. The method of claim 18, wherein said target tissue is within a patient.

29. The method of claim 28, wherein said contacting comprises administering said microbubble to said patient.

30. The method of claim 18, wherein said imaging creates an X-ray dark field image of said target tissue.

31. The method of claim 18, wherein said at least one metal nanoparticle is a metal nanoparticle layer.

32. A composition, comprising a dual shell polymer microbubble comprising an inner polymer layer and an outer polymer layer, wherein said outer polymer layer comprises at least one metal nanoparticle.

33. The composition of claim 32, wherein said dual shell polymer further comprises a gas-filled hollow core.

34. The composition of claim 32, wherein said outer polymer layer further comprises an amphiphilic biocompatible material.

35. The composition of claim 32, wherein said at least one metal nanoparticle is a metal nanoparticle layer.

36. The composition of claim 32, wherein said at least one metal nanoparticle is covalently attached to said outer polymer layer.

37. The composition of claim 32, wherein said at least one metal nanoparticle is completely embedded within said outer polymer layer.

38. The composition of claim 32, wherein said at least one metal nanoparticle is selected from the group consisting of gold nanoparticle and at least one iron oxide nanoparticle.

39. The composition of claim 33, wherein said gas-filled hollow core comprises a gas selected from the group consisting of air and nitrogen.

40. The composition of claim 34, wherein said amphiphilic biocompatible material is a blood compatible material.

41. The composition of claim 32, wherein said inner polymer layer comprises at least one biodegradable polymer.

42. The composition of claim 32, wherein said outer polymer layer comprises at least one biodegradable polymer.
